(19) United States
(12) Patent Application Publication
(10) Pub. No.: US 2025/0318979 A1
(43) Pub. Date: Oct. 16, 2025

(54) METHOD AND SYSTEM FOR IMPLEMENTING VISION TRAINING

(71) Applicant: Orthov Technology Company Limited, Kaohsiung City (TW)

(72) Inventors: Hsuan-Yu HUANG, Kaohsiung City (TW); Ping-Hsu CHENG, Kaohsiung City (TW)

(73) Assignee: Orthov Technology Company Limited, Kaohsiung City (TW)

(21) Appl. No.: 19/176,890

(22) Filed: Apr. 11, 2025

(30) Foreign Application Priority Data
Apr. 15, 2024 (TW) 113114014

(51) Int. Cl. A61H 5/00; A61B 3/00

(52) U.S. Cl. CPC A61H 5/00; A61B 3/0025; A61B 3/005

(57) ABSTRACT

A method for implementing vision training is implemented using a portable electronic device and a vision device that includes two optical units that are arranged in front of two eyes of the user, respectively. The method includes: displaying an icon on the portable electronic device to enable the user to see through the vision device a clear image of the icon; controlling the optical units to dynamically adjust a physics variable; in response a user-input termination command indicating that the user sees two blurry images of the icon, determining a current physics variable of each of the optical units; calculating a test score based on the current physics variables of the optical units; and initiating a vision training session that includes playing a video source and controlling the optical units to adjust the physics variable in one of a refractive error process and a recovery process.

### CROSS-REFERENCE TO RELATED APPLICATION

This application claims priority to Taiwanese Invention Patent Application No. 113114014, filed on Apr. 15, 2024, the entire disclosure of which is incorporated by reference herein.

### FIELD

The disclosure relates to a method and a system for implementing vision training, and more particularly to a method and a system for implementing a vision training operation on extraocular muscles and/or optic nerves of a user.

### BACKGROUND

As the use of electronic devices becomes popular in the daily life, people tend to stare at electronic displays for an extended period. One apparent drawback from the intense of use of the eyes is eye fatigue, which may result in different symptoms (e.g., myopia, amblyopia, vergence dysfunction, etc.) that adversely affect the vision. It is noted that eye fatigue is typically resulted from the overuse of the eyes, which causes issues to the extraocular muscles, the ciliary muscles and/or optic nerves of a user.

In order to address the symptoms related to the vision, some methods and equipment have been developed to implement various vision training. For example, Chinese Invention Patent No. CN111929897B discloses a virtual reality (VR) equipment for ciliary muscle exercise to be worn by a user for vision training (specifically, for myopia treatment). The VR equipment is typically embodied in the form of a headset, includes an equipment main body, a screen disposed on the equipment main body for displaying objects, a convex lens that is disposed on the equipment main body, and a step motor disposed on the equipment main body. The screen may be controlled by the step motor to move within a plurality of sub motion ranges on a sliding rail, and the movement of the screen changes a distance between the screen and the convex lens. When the VR equipment is worn by a user, by controlling the movement of the screen repeatedly, the ciliary muscles of the user is compelled to stretch and contract continuously, and the symptom of myopia may be improved.

It is noted that the VR equipment includes a number of components (i.e., the equipment main body, the screen, the convex lens and the step motor) which may cause the weight of the VR equipment to increase to the point that causes discomfort to the user wearing the VR equipment. In addition, by disposing the screen on the equipment main body, the screen becomes relatively close to the eyes of the user. In the case where the screen is configured to display an object with a relatively higher brightness, the resulting effect on the eyes of the user may cause serious eye diseases such as glaucoma, macular degeneration, etc.

Additionally, the operations of the VR equipment typically require user information related to the eye conditions of the user, and the user usually needs to input the user information manually. In the case where the user is a young child, input of the user information may be difficult.

Moreover, the VR equipment is specifically designed for training the ciliary muscles of the user, and may encounter other issues. For example, the selection of the objects to be displayed on the screen may be limited, and/or the objects may not be interesting for the users.

### SUMMARY

Therefore, one object of the disclosure is to provide a method for implementing vision training that can alleviate at least one of the drawbacks of the prior art.

According to one embodiment of the disclosure, the method for implementing vision training is implemented using a portable electronic device that is separate from and in communication with a vision device. The vision device is worn by a user and includes two optical units that are arranged in front of two eyes of the user, respectively. The method includes:
a) displaying an icon on a touchscreen of the portable electronic device to enable the user to see through the vision device a clear image of the icon on the touchscreen;
b) for each of the optical units, controlling the optical unit to dynamically adjust a physics variable of the optical unit incrementally during a preset time period, the physics variable of each of the optical units indicating an extent to which a light ray passing through the optical unit being deflected;
c) in response to generation of a user-input termination command by the touchscreen indicating that the user sees two blurry images of the icon, controlling each of the optical units to stop adjusting the physics variables of the optical units, and determine a current physics variable of each of the optical units;
d) calculating a test score based on the current physics variables of the optical units; and
e) in response to receipt of a training command, calculating a set of training parameters to be employed during a vision training session, and initiating the vision training session that includes
playing a video source on the touchscreen, and
controlling each of the optical units to adjust the physics variable of the optical unit based on the set of training parameters in one of a refractive error process, in which the user sees through the vision device blurry images of the video source on the touchscreen, and a recovery process, in which the user sees through the vision device clear images of the video source on the touchscreen.

Another object of the disclosure is to provide a system that is configured to implement the above-mentioned method.

According to one embodiment of the disclosure, the system for implementing vision training includes a vision device to be worn by a user, and a portable electronic device that is separate from and in communication with the vision device.

The vision device including two optical units that are arranged in front of two eyes of the user, respectively. The optical units are operable to dynamically adjust physics variables of the optical units. The physics variable of each of the optical units indicates an extent to which a light ray passing through the optical unit being deflected.

The portable electronic device includes a touchscreen and a processor that is configured to implement steps of the method as claimed in claim 1.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a schematic view of a system for implementing vision training according to one embodiment of the disclosure.

FIG. 2 is a block diagram illustrating the components of the system according to one embodiment of the disclosure.

FIG. 3 is a perspective view of an exemplary vision device according to one embodiment of the disclosure.

FIG. 4 is a partial diagram of the system illustrating the optical units according to one embodiment of the disclosure, showing one clear image.

FIG. 5 is a partial diagram of the system illustrating the optical units according to one embodiment of the disclosure, showing two blurry sub-images.

FIG. 6 is a flow chart illustrating steps of a method for implementing vision training according to one embodiment of the disclosure.

FIG. 7 illustrates an exemplary GUI according to one embodiment of the disclosure.

FIG. 8 illustrates an exemplary GUI according to one embodiment of the disclosure.

FIG. 9 illustrates is a partial diagram illustrating the optical units according to one embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

Throughout the disclosure, the term “coupled to” or “connected to” may refer to a direct connection among a plurality of electrical apparatus/devices/equipment via an electrically conductive material (e.g., an electrical wire), or an indirect connection between two electrical apparatus/devices/equipment via another one or more apparatus/devices/equipment, or wireless communication.

It should be noted herein that for clarity of description, spatially relative terms such as “top,” “bottom,” “upper,” “lower,” “on,” “above,” “over,” “downwardly,” “upwardly” and the like may be used throughout the disclosure while making reference to the features as illustrated in the drawings. The features may be oriented differently (e.g., rotated 90 degrees or at other orientations) and the spatially relative terms used herein may be interpreted accordingly.

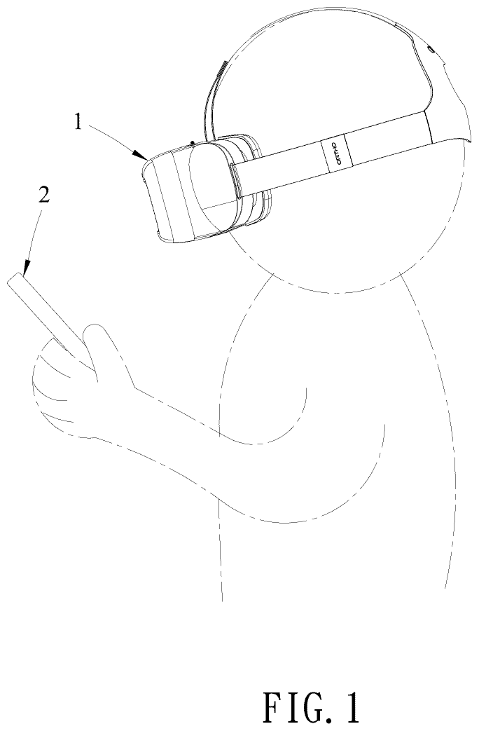

FIG. 1 is a schematic view of a system for implementing vision training according to one embodiment of the disclosure. In this embodiment, the system includes a vision device 1 and a portable electronic device 2. The vision device 1 is to be worn by a user. The portable electronic device 2 may be held by the user or placed on a surface (e.g., on a table).

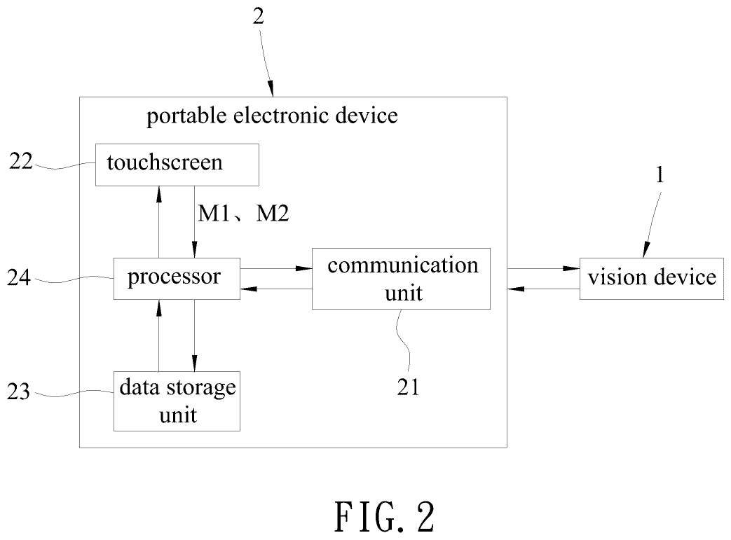

FIG. 2 is a block diagram illustrating the components of the system according to one embodiment of the disclosure. It is noted that in embodiments, the vision device 1 is remote from the portable electronic device 2, and is in a wireless communication with the portable electronic device 2.

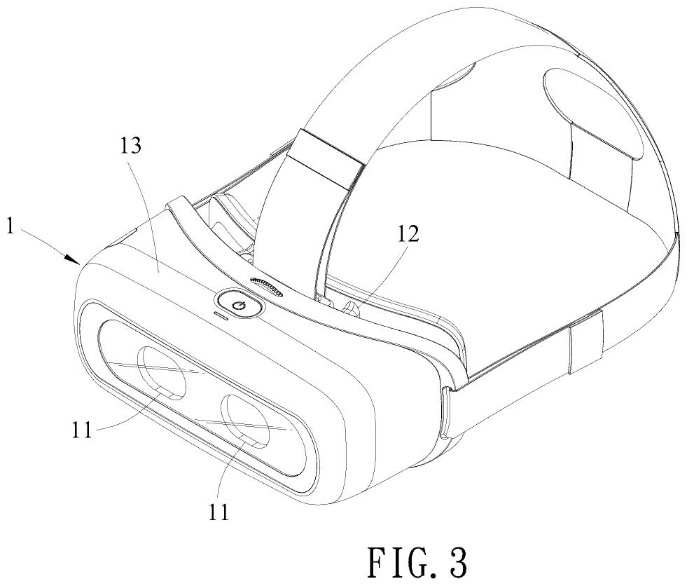

FIG. 3 is a perspective view of an exemplary vision device 1 according to one embodiment of the disclosure. The vision device 1 is embodied using a headset in the embodiment of FIG. 3, and includes two optical units 11, a controller 12 and a communication unit 13 electrically connected to the controller 12. The controller 12 is coupled to the two optical units 11, and includes a motor and a motor driver (not shown) for changing configurations of the optical units 11.

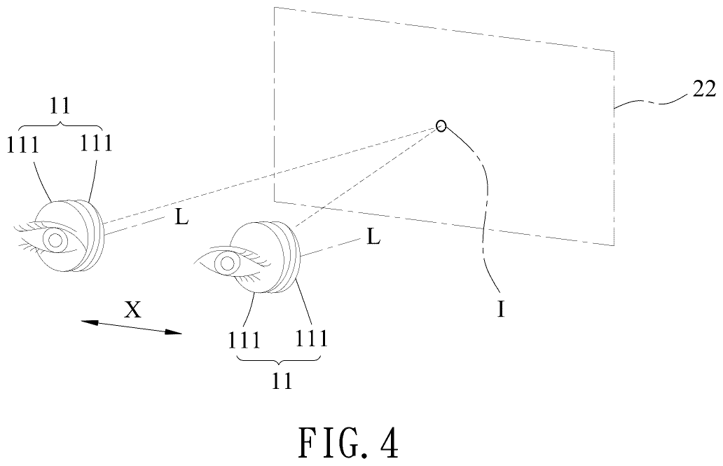

FIG. 4 is a partial diagram of the system illustrating the optical units 11 according to one embodiment of the disclosure. Each of the two optical units 11 includes two optical prisms 111. In the embodiment of FIG. 4, the two optical units 11 are spaced apart from each other along a horizontal direction (label as X on FIG. 4), and the two optical prisms 111 of each of the optical units 11 are arranged along a respective one of axes (labeled as L1 and L2 on FIG. 4) of the optical units 11. That is to say, when the vision device 1 is worn by the user, the optical units 11 are arranged in front of the two eyes of the user, respectively, and the user is able to see through the optical units 11.

Each of the two optical units 11 is configured to alter the light passing therethrough by a physics variable. Generally, the physics variable of each of the optical units 11 indicates an extent to which a light ray passing through the optical unit 11 is deflected.

In some embodiments, the physics variable is a prism diopter Δ. A definition of one unit of the prism diopter Δ is defined such that with respect to one optical unit 11 with the prism diopter 1*Δ, when a light ray originated from an infinite distance enters one side of the optical prisms 111 of the optical unit 11 and passes through the optical prisms 111 along a corresponding optical axis, the light ray is deflected such that a resulting imaging at a position one meter away from the other side of the optical prisms 111 of the optical unit 11 is one centimeter that deviates from the optical axis.

Each of the optical prisms 111 includes a base (which may be a thicker part of the optical prism), and may be controlled to switch between a lateral rotating state and a vertical rotating state.

In the lateral rotating state, the two optical prisms 111 of each of the optical units 11 may be disposed at an initial rotating location, at which the base of one of the optical prisms 111 faces upward and the base of the other one of the optical prisms 111 faces downward (i.e., vertical positions), and for each of the optical units 11, the prism diopter 1 is zero. For each of the optical units 11, each of the optical prisms 111 is controlled to rotate with respect to the corresponding axis L1 or L2. Based on rotational directions of the optical prisms 111, the rotations of the optical prisms 111 may be called one of a base in (BI) mode, in which the bases of the optical prisms 111 rotate toward a nose of the user (i.e., toward a lateral position), and a base out (BO) mode, in which the bases of the optical prisms 111 rotate away from the nose of the user (i.e., toward another lateral position).

In the vertical rotating state, the two optical prisms 111 of each of the optical units 11 may be disposed at an initial rotating location, at which the base of one of the optical prisms 111 faces left and the other one of the bases of the optical prisms 111 faces right (i.e., the lateral positions), and for each of the optical units 11, the prism diopter Δ is zero. The base of each of the optical prisms 111 of each of the optical units 11 is controlled to rotate with respect to the corresponding axis L1 or L2. Based on rotational directions of the optical prisms 111, the rotations of the optical prisms 111 may be called one of a base up (BU) mode, in which the bases of optical prisms 111 rotate upward (i.e., toward the vertical position), and a base down (BD) mode, in which the bases of the optical prisms 111 rotate downward (i.e., toward the other vertical position).

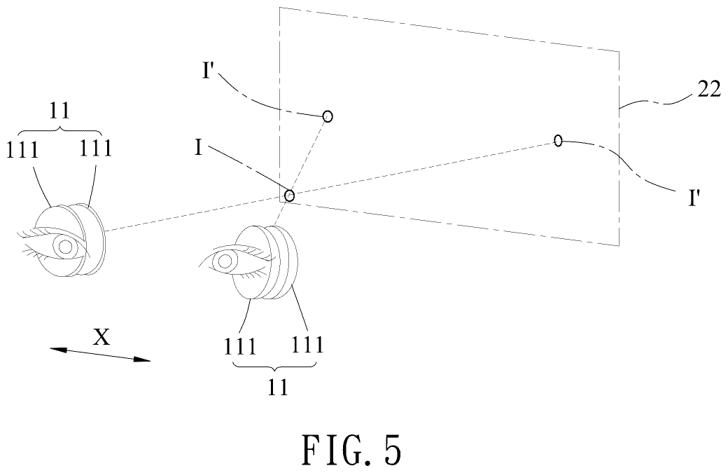

Using the above operations, the prism diopter Δ associated with each of the optical units 11 may be adjusted via physical changes. In some embodiments, the prism diopter Δ associated with each of the optical units 11 may be represented using a number, preceded by a plus sign (+) in the BO mode or the BU mode, or a minus sign (−) in the BI mode or the BD mode. By operating each of the optical units 11, a resulting optical imaging on a surface to be seen by the eyes of the user may be a clear image (labeled as I) or two blurry sub-images (labeled as I′) that are spaced apart from each other based on the corresponding prism diopter Δ. FIG. 5 is a partial diagram of the system illustrating the optical units 11 according to one embodiment of the disclosure, showing two blurry sub-images I′.

For example, in the case where an optical unit 11 is configured in the BI mode to rotate, and both of the optical units 11 have the same prism diopter Δ of −1, the light rays passing through the optical units 11 are deviated away from the respective optical axes by one unit along the horizontal direction toward the nose. In response, the user seeing through the optical units 11 would turn his/her eyes outward. In the case where an optical unit 11 is configured in the BO mode to rotate, and both of the optical units 11 have the same prism diopter Δ of +2, the light rays passing through the optical units 11 are deviated away from the respective optical axes by two units along the horizontal direction toward the respective temples. In response, the user seeing through the optical units 11 would turn his/her eyes inward.

It is noted that the operations and structures related to forming the optical units 11 and rotating the optical prisms 111 are readily known in the related art. For example, Taiwanese Invention Patent No. TWI781072B discloses a visual inspection and training device that includes the relevant structures. As such, details thereof are omitted herein for the sake of brevity.

In the embodiment of FIG. 1, the portable electronic device 2 may be embodied using a tablet, a smartphone, a laptop, or other suitable electronic devices. The portable electronic device 2 includes a communication unit 21, a touchscreen 22, a data storage unit 23, and a processor 24 as shown in FIG. 2.

The processor 24 is connected to the communication unit 21, the touchscreen 22 and the data storage unit 23, and may be embodied using one or more of a central processing unit (CPU), a microprocessor, a microcontroller, a single core processor, a multi-core processor, a dual-core mobile processor, a microprocessor, a microcontroller, a digital signal processor (DSP), a field-programmable gate array (FPGA), an application-specific integrated circuit (ASIC), a radio-frequency integrated circuit (RFIC), etc.

The communication unit 21 may include one or more of a radio-frequency integrated circuit (RFIC), a short-range wireless communication module supporting a short-range wireless communication network using a wireless technology of Bluetooth® and/or Wi-Fi, etc., and a mobile communication module supporting telecommunication using Long-Term Evolution (LTE), the third generation (3G) of, the fourth generation (4G) of or the fifth generation (5G) of wireless mobile telecommunications technology, or the like. The communication unit 21 is configured to establish a wireless communication to the communication unit 13 of the vision device 1. The communication unit 13 may be implemented in the same way as the communication unit 12.

The data storage unit 23 may be embodied using, for example, one or more of random access memory (RAM), read only memory (ROM), programmable ROM (PROM), firmware, flash memory, a hard disk drive (HDD), etc. In some embodiments, the data storage unit 23 may be hardware components that are built in the portable electronic device 2 or that are externally connected to the portable electronic device 2. The data storage unit 23 may store a software application that includes instructions that, when executed by the processor 24, cause the processor 24 to implement the operations as described below. In some embodiments, the software application may be a vision training application.

The touchscreen 22 is configured to be controlled by the processor 24 to display content thereon, and to serve as a user interface to enable the user to interact with the portable electronic device 2 by, for example, displaying a graphical user interface (GUI) thereon or receiving a user input.

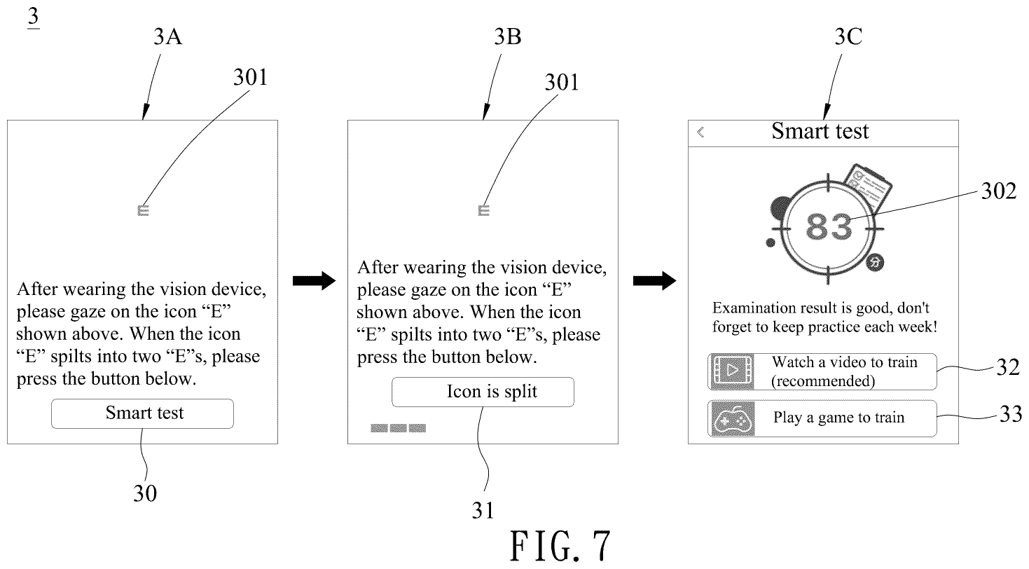

FIG. 7 illustrates an exemplary GUI 3 according to one embodiment of the disclosure. In the embodiment of FIG. 7, the GUI 3 may be generated after the user operates the portable electronic device 2 to execute the vision training application and selects an eye testing procedure, and includes texts, icons, image and/or buttons.

In a first screen 3A, at the start of the eye testing procedure, the GUI 3 includes an icon 301 that may be a character used for an eye test, texts for instructing the user, and a first button 30. In the embodiment of FIG. 7, the instruction of the first screen 3A tells the user to wear the vision device 1, press the button 30, stare at the icon 301, and press another button when he/she sees the icon 301 split in two. After the user presses the button 30, the processor 12 of the vision device 1 may be controlled to actuate the optical prisms 111 of the optical units 11 to rotate, so as to cause the two light rays entering the optical units 11 to deviate away from each other, which in turn causes the icon 301 seen by the user wearing the vision device 1 to start “splitting.” In a second screen 3B, a button 31 is displayed, which enables the user to press the button 31 when the icon 301 is deemed to split in two. Afterwards, the processor 24 may calculate a test score based on a time at which the user presses the button 31, and display an image 302 indicating the test score on a subsequent third screen 3C.

The third screen 3C may further include other buttons that are linked to other functions of the vision training application. For example, in the embodiment of FIG. 7, the third screen 3C may further include a button 32 that is associated with a video training function, and a button 33 that is associated with a game playing training function.

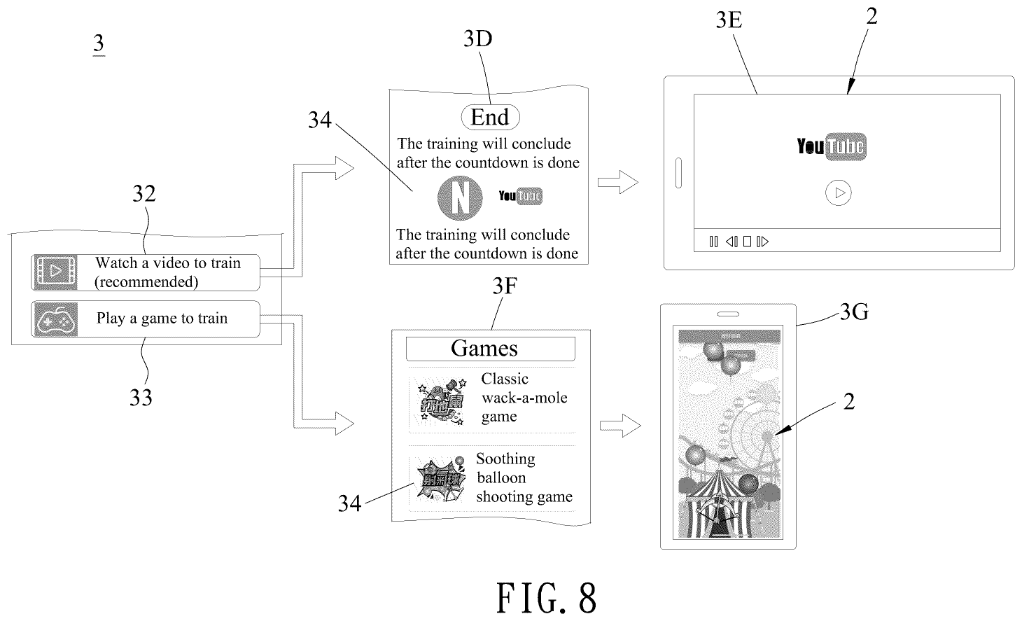

FIG. 8 illustrates an exemplary GUI 3 according to one embodiment of the disclosure. Specifically, after the user presses the button 32, a fourth screen 3D may be displayed, which may include a number of links associated with different video platforms (e.g., Netflix, YouTube®, etc.) on the fourth screen 3D. After the user selects one of the video platforms, the processor 24 may execute an application or open a web browser to display a website associated with the selected one of the video platforms to play a video for training on a fifth screen 3E. That is to say, the portable electronic device 2 obtains the video for training from a cloud storage. In some other embodiments, a training video may be pre-stored in the data storage unit 23. Alternatively, after the user presses the button 33, a sixth screen 3F may be displayed, which may include a number of links associated with different games (e.g., a whack-a-mole game, a balloon shooting game, etc.). After the user selects one of the games, the processor 24 may execute another application associated with the selected one of the games to enable the user to play on a seventh screen 3G for training.

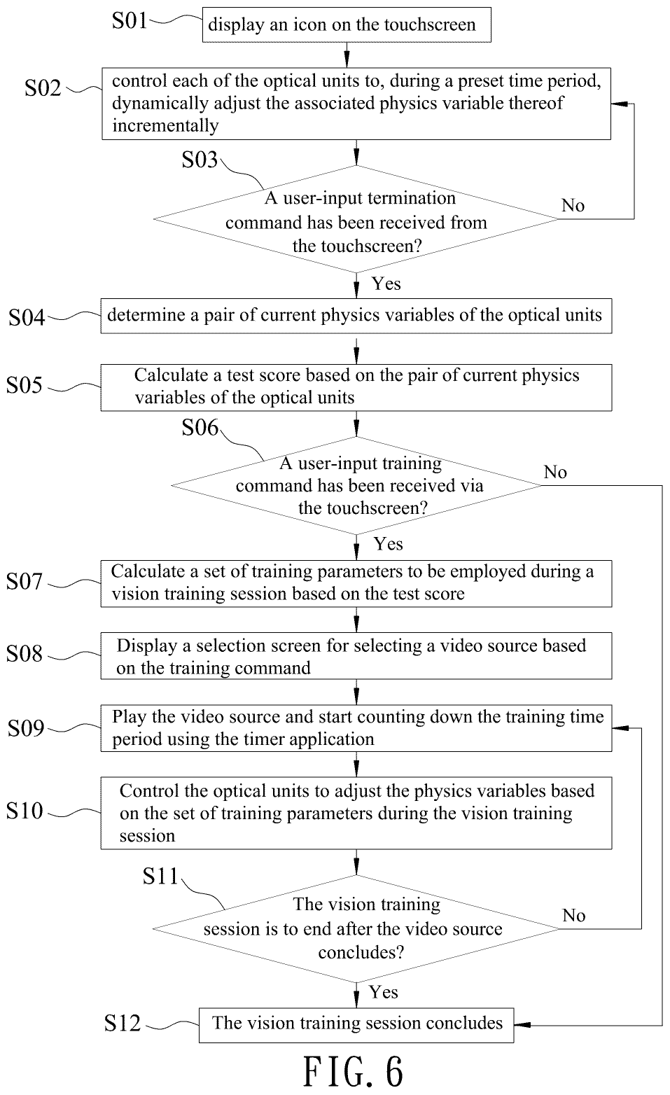

FIG. 6 is a flow chart illustrating steps of a method for implementing vision training according to one embodiment of the disclosure. In the embodiment of FIG. 6, the method is implemented using the system as shown in FIG. 2.

In use, after the user wears the vision device 1 and holds the portable electronic device 2 (as shown in FIG. 1), the user may operate the portable electronic device 2 to execute the software application (using, for example, the touchscreen 22), and in turn, the processor 24 causes the system to implement the steps of the method of FIG. 6 as shown below.

In step S01, the processor 24 controls the touchscreen 22 to display an icon 301 (see FIG. 7), so that the user wearing the vision device 1 is able to see a clear image (I) of the icon on the touchscreen 22 (see FIG. 4). In use, the touchscreen 22 may be controlled to display the first screen 3A, with the text instructions and the button 30. In embodiments, the user may be instructed to place the portable electronic device 2 in front of his/her eyes at a predetermined distance, such as 30 to 40 centimeters.

In step S02, the processor 24 controls each of the optical units 11 of the vision device 1 to, during a preset time period, dynamically adjust the associated prism diopters Δ thereof incrementally. It is noted that the operations for adjusting the prism diopters Δ may be done using the manners as described above. In use, after the user pushes the button 30, the processor 24 controls the touchscreen 22 to display the second screen 3B, with the text instructions and the button 31. In use, the preset time period may be about 5 to 30 seconds, and may be counted by the processor 24 executing a timer application.

In one implementation, the prism diopter Δ of each of the optical units 11 may be initially +1.0 Δ. During the operation of step S02, the prism diopter Δ of each of the optical units 11 may be controlled to increase gradually by a fixed increment (e.g., +0.1 Δ) to a target value (e.g., +40.0 Δ). That is to say, the prism diopter Δ of each of the optical units 11 may be adjusted from +1.0 Δ to, sequentially, +1.1 Δ, +1.2 Δ, +1.3 Δ, . . . , +1.1 Δ, +39.9 Δ, and eventually +40.0 Δ.

As such, to the user wearing the vision device 1, the dynamic changes of the prism diopter Δ of each of the optical units 11 cause the light rays traveling through the optical units 11 to be deflected in a way the resulting image of the icon 301 on the touchscreen 22 being “split” into two sub-images (I′) that are away from each other as seen by the user. FIG. 5 shows two such sub-images (I′) that are spaced apart horizontally.

It is noted that the initial prism diopter Δ of each of the optical units 11, the fixed increment and the target value are not limited to the numbers as described above. Additionally, due to the different operations (e.g., the vertical rotating state) used to adjust the optical units 11, the image of the icon 301 on the touchscreen 22 may also be split into two sub-images (I′) that are spaced apart vertically.

During the operations of step S02, in step S03, the processor 24 determines whether a user-input termination command M1 has been received from the touchscreen 22. In the case where it is determined that the termination command M1 has been received, the processor 24 controls the optical units 11 of the vision device 1 to stop adjusting the associated prism diopters Δ thereof, and the flow proceeds to step S04. Otherwise, the flow goes back to step S02.

It is noted that in embodiments, the user is instructed to, when the user sees the image on the touchscreen 22 spilt into two sub-images (I′), press the button 31. In response to the press of the button 31, the termination command M1 is generated and transmitted to the processor 24.

In step S04, the processor 24 determines a pair of current physics variables of the optical units 11. That is to say, the processor 24 determines a current prism diopter Δ of each of the optical units 11, based on controlling of and a mode of operation of each of the optical units 11.

In one example, the processor 24 determines that the current prism diopter Δ of each of the optical units 11 is −2.0Δ, and each of the optical units 11 is controlled to operate in the BI mode. That is to say, it may be determined that the optical units 11 have rotated in the BI mode by 2.0Δ.

Afterward, in step S05, the processor 24 calculates a test score based on the current prism diopter Δ of each of the optical units 11. The test score may be calculated using existing methods, and may be then displayed in the image 302. In some examples, the test score may be 2.5 times of the current prism diopter Δ.

In step S06, the processor 24 determines whether a user-input training command M2 has been received via the touchscreen 22. In the case where it is determined that the training command M2 has been received, the flow proceeds to step S07. Otherwise, the flow proceeds to step S12 in which the method is terminated.

It is noted that in embodiments, when the user presses the button 32 or the button 33, the training command M2 is generated and transmitted to the processor 24.

In step S07, in response to receipt of the training command M2, the processor 24 calculates a set of training parameters to be employed during a vision training session based on the test score. In the embodiment of FIG. 6, the set of training parameters may include an operation mode for the optical units 11, a number of target physics variables that define a range, and a training time period.

In one example, the operation mode may be the BI mode or the BO mode, the target physics variables are −4.0Δ and +8.0Δ, and the training time period is 10 or 15 minutes, but in other embodiments, other training parameters may be employed.

In step S08, the processor 24 controls the touchscreen 22 to display a selection screen for selecting a video source based on the training command M2. For example, in the case where the user presses the button 32 in step S06, the processor 24 controls the touchscreen 22 to display the fourth screen 3D for user's selection of a video platform or the data storage 23, and for user's selection of a video from the selected video platform or the data storage 23 to serve as the video source. Alternatively, in the case where the user presses the button 33 in step S06, the processor 24 controls the touchscreen 22 to display the sixth screen 3F for user's selection of a game to serve as the video source.

In step S09, after the user selects the video source, the processor 24 controls the touchscreen 22 to play the video source (i.e., to start the vision training session), and starts counting down the training time period using the timer application.

In step S10, the processor 24 controls the optical units 11 to adjust the physics variables based on the set of training parameters during the vision training session.

Specifically, in some embodiments, the vision training session may be implemented in one or both of a refractive error process and a recovery process. The refractive error process is designed to cause the user to see the image (I) being blurry and split into two sub-images (I′) that move away from each other. As such, the target physics variables may be +nΔ and +40Δ, where +nΔ represents the current prism diopter Δ. For example, in the case where the current prism diopter Δ is +4, the target physics variables may be +4Δ and +40Δ, or other suitable combinations. The recovery process is designed to cause the user to see the image (I) being clear. As such, the target physics variables may be 0Δ and −21Δ, or other suitable combinations.

As such, while the user is watching the video or playing the game during the vision training session, the processor 24 controls the optical units 11 to operate in one or both of the refractive error process and the recovery process.

In the refractive error process, the processor 24 controls the optical units 11 to adjust the physics variables to a fixed value between the target physics variables (i.e., +4Δ and +40Δ). It is noted that the refractive error process is designed such that the user sees split blurry images of the video source on the touchscreen 22.

Alternatively, the processor 24 may control the optical units 11 such that the physics variables of the optical units 11 change from one to the other of the target physics variables (i.e., +4Δ and +40Δ) in an oscillating manner. Specifically, at the start of the refractive error process, the physics variables of the optical units 11 are +4Δ. During the refractive error process, the processor 24 controls the optical units 11 such that the physics variables of the optical units 11 change from +4Δ gradually by a fixed increment to +40Δ. Afterward, the processor 24 controls the optical units 11 such that the physics variables of the optical units 11 change gradually by a fixed decrement to +4Δ. The above operations are then repeated during the refractive error process.

In the recovery process, the processor 24 controls the optical units 11 to adjust the physics variables to a fixed value between the target physics variables (i.e., 0Δ and −21Δ). It is noted that the recovery process is designed such that the user sees clear images of the video source on the touchscreen 22.

Alternatively, the processor 24 may control the optical units 11 such that the physics variables of the optical units 11 change from one to the other of the target physics variables (i.e., 0Δ and −21Δ) in the oscillating manner. Specifically, at the start of the recovery process, the physics variables of the optical units 11 are 0Δ. During the recovery process, the processor 24 controls the optical units 11 such that the physics variables of the optical units 11 change from 0Δ gradually by a fixed decrement to −21Δ. Afterward, the processor 24 controls the optical units 11 such that the physics variables of the optical units 11 change gradually by the fixed increment back to 0Δ. The above operations are then repeated during the recovery process.

It is noted that in some embodiments, one vision training session may include both a refractive error process and a recovery process, or multiple refractive error processes and multiple recovery processes in an alternating manner.

For example, multiple refractive error processes may be performed in the alternating manner for a time period ranging from 10 to 15 minutes. In each repeated cycle of the multiple refractive error processes, the optical units 11 initially operate in the BO mode, where the physics variables gradually change from 0Δ to +8.0Δ and then back to 0Δ, and then proceed to the BI mode, where the target physics variables gradually change from 0Δ to −4.0Δ and then back to 0Δ.

In step S11, after the video source concludes (i.e., the video selected has been played in entirety or the game has ended), the processor 24 determines whether the vision training session is to end. In use, as soon as the vision training session is initiated, the processor 24 may execute the timer application. In the case where it is determined that the training time period (10-15 minutes) has elapsed, the vision training session concludes, and the method is terminated (i.e., step S12). Otherwise, the flow goes back to step S09 where the processor 24 controls the touchscreen 22 to play a next video source selected by the user.

In short, by controlling the physics variables of the optical units 11 to change back and forth gradually by the fixed increment and the fixed decrement, and by alternating between the refractive error process and the recovery process, the extraocular muscles and the ciliary muscles of the user may be compelled to stretch and contract, therefore potential eye fatigue related to the extraocular muscles and the ciliary muscles and/or optic nerves of the user may be reduced.

In some embodiments, the physics variables of the optical units 11 may be other properties. For example, by controlling the optical prisms 111 to rotate, a focal length of each of the optical units 11 may also be adjusted to serve as a physics variable. In some embodiments, by controlling the optical prisms 111 to rotate, a combination of the focal length and the prism diopter of each of the optical units 11 may also be adjusted to serve as a physics variable.

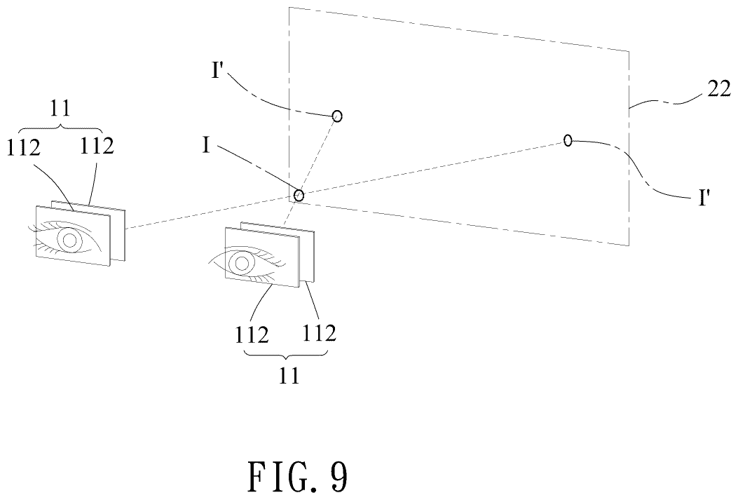

FIG. 9 is a partial diagram illustrating the optical units 11 according to one embodiment of the disclosure. In the embodiment of FIG. 9, each of the optical units 11 includes two optical screens 112 that are disposed in parallel. The optical screens 112 may be embodied using liquid crystal (LC) screens that include LC molecules therein. As such, the vision device 1 may be considered as including a pair of LC lenses, and may resemble ordinary eyeglasses in appearance. In use, by arranging molecular orientations of the LC molecules using an external electric field, the optical units 11 may be configured to deflect light in a desired manner (i.e., to adjust the physics variables). As such, the vision device 1 as shown in FIG. 9 is capable of achieving similar effects of the aforementioned embodiments.

In sum, embodiments of the disclosure provide a method and a system for implementing vision training. The method and the system provide at least the following advantages.
1) The system includes a vision device 1 and a separate portable electronic device 2, and thus has the advantage that the vision device 1, which is to be worn by the user, may have a reduced weight since some or more of the components may be omitted. Additionally, the portable electronic device 2, on which the icon 301 and the video source are displayed, may be placed at a greater distance from the eyes of the user compared with the conventional VR headsets. As a result, the potential symptoms associated with usage of the system such as glaucoma, macular degeneration, etc. may be reduced.
2) The system provides a GUI 3 on the portable electronic device 2 that enables the user to operate with relative ease, without having to manually input the user information by operating the vision device 1.
3) The system provides a vision training session that includes displaying a video source on the touchscreen 22. The video source may be a video stream from an online video platform, a video stored in the portable electronic device 2 or a video game selected by the user. The video may be pre-stored in the portable electronic device 2 or obtained from an online source and stored in the data storage unit 23. As such, the vision training session may be conducted with the user viewing his/her preferred content, which is a marked improvement over the conventional vision training technique.
4) The portable electronic device 2 may be embodied using a tablet, a smartphone, a laptop, etc., which are commonly available hardware. Therefore, the user only needs to purchase the vision device 1 which is less costly.
5) During the vision training session, the physics variables of the optical units 11 are controlled to change gradually by the fixed increment and/or the fixed decrement. As such, the user may experience the changes of the physics variables during the vision training session with more comfort.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for implementing vision training, the method being implemented using a portable electronic device that is separate from and in communication with a vision device, the vision device being worn by a user and including two optical units that are arranged in front of two eyes of the user, respectively, the method comprising:
a) displaying an icon on a touchscreen of the portable electronic device to enable the user to see through the vision device a clear image of the icon on the touchscreen;
b) for each of the optical units, controlling the optical unit to dynamically adjust a physics variable of the optical unit incrementally during a preset time period, the physics variable of each of the optical units indicating an extent to which a light ray passing through the optical unit being deflected;
c) in response to generation of a user-input termination command by the touchscreen indicating that the user sees two blurry images of the icon, controlling each of the optical units to stop adjusting the physics variables of the optical units, and determine a current physics variable of each of the optical units;
d) calculating a test score based on the current physics variables of the optical units; and
e) in response to receipt of a training command, calculating a set of training parameters to be employed during a vision training session, and initiating the vision training session that includes
playing a video source on the touchscreen, and
controlling each of the optical units to adjust the physics variable of the optical unit based on the set of training parameters in one of a refractive error process, in which the user sees through the vision device blurry images of the video source on the touchscreen, and a recovery process, in which the user sees through the vision device clear images of the video source on the touchscreen.

2. The method as claimed in claim 1, wherein the video source is selected by the user and includes one of a video stream from an online video platform, a video stored in the portable electronic device, and a video game.

3. The method as claimed in claim 2, wherein the video is pre-stored in the portable electronic device or is obtained from an online source and stored in the portable electronic device.

4. The method as claimed in claim 1, wherein in step b), dynamic changes of the physics variable of each of the optical units cause the light rays traveling through the optical units to be deflected in a way a resulting image of the icon on the touchscreen splits into two sub-images away from each other as seen by the user.

5. The method as claimed in claim 4, wherein in step e):
in the refractive error process, the physics variable of each of the optical units is set such that the user sees two split blurry images of the video source on the touchscreen; and
in the recovery process, the physics variable of each of the optical units is set such that the user sees one clear image of the video source on the touchscreen.

6. The method as claimed in claim 4, wherein:
the physics variable of each of the optical units is a prism diopter;
in the refractive error process, the physics variable of each of the optical units is set based on a range from (n) to 40, wherein (n) represents the current physics variable; and
in the recovery process, the physics variable of each of the optical units is set based on a range from 0 to −21.

7. The method as claimed in claim 4, wherein the two sub-images related to the icon are spaced apart from each other horizontally or vertically.

8. The method as claimed in claim 7, wherein the physics variable of each of the optical units is a prism diopter.

9. A system for implementing vision training, comprising:
a vision device to be worn by a user, the vision device including two optical units that are arranged in front of two eyes of the user, respectively, the optical units being operable to dynamically adjust physics variables of the optical units, the physics variable of each of the optical units indicating an extent to which a light ray passing through the optical unit being deflected; and
a portable electronic device that is separate from and in communication with the vision device, the portable electronic device including a touchscreen and a processor that is configured to implement steps of the method as claimed in claim 1.

10. The system as claimed in claim 9, wherein the portable electronic device is one of a tablet, a smartphone, and a laptop.

11. The system as claimed in claim 9, wherein:
each of the optical units includes two optical prisms arranged along a respective one of axes of the optical units; and
for each of the optical units, each of the optical prisms includes a base, and is controlled to switch between a lateral rotating state, in which the optical prism is controlled to rotate with respect to a corresponding axis and the base rotates from a vertical position toward a lateral position, and a vertical rotating state, in which the optical prism is controlled to rotate with respect to the corresponding axis and the base rotates from the lateral positon toward the vertical position, so as to adjust the physics variables of the optical units.

12. The system as claimed in claim 9, wherein:
each of the optical units includes two liquid crystal (LC) screens that are disposed in parallel, the LC screens including LC molecules therein; and
molecular orientations of the LC molecules are capable of being arranged using an external electric field to make the optical units deflect light in a desired manner.
